(19) United States
(12) Patent Application Publication
(10) Pub. No.: US 2026/0272338 A1
(43) Pub. Date: Sep. 17, 2026

(54) MEDICAL DEVICE AND METHOD OF ASSEMBLING

(71) Applicant: Roche Diabetes Care GmbH, Mannheim (DE)

(72) Inventor: Olaf Roscher, Mannheim (DE)

(21) Appl. No.: 19/664,460

(22) Filed: Apr. 30, 2026

Related U.S. Application Data
(63) Continuation of application No. PCT/EP2024/080700, filed on Oct. 30, 2024.

(30) Foreign Application Priority Data
Nov. 2, 2023 (EP) 23 207 430.2

(51) Int. Cl. A61B 5/145; A61B 5/00

(52) U.S. Cl. CPC A61B 5/14546; A61B 5/6847

(57) ABSTRACT

A medical device for detecting at least one physiological parameter of a user has an electronics unit and an analytical sensor configured for transdermal insertion into body tissue of a user. The sensor has an insertable portion configured for at least partially being inserted into the body tissue and an electrical connection portion electrically connected to the electronics unit. A housing made of a housing material receives the electronics unit and at least the insertable portion protrudes from an opening in the housing. A guide at least partially made of a guiding material softer than the housing material at least partially surrounds the analytical sensor at the opening. A medical system with the medical device, and a method of assembling the medical device are also disclosed.

### RELATED APPLICATIONS

This application is a continuation of PCT/EP 2024/080700, filed Oct. 30, 2024, which claims priority to EP 23 207 430.2, filed Nov. 2, 2023, the entire disclosures of both of which are hereby incorporated herein by reference.

### BACKGROUND

This disclosure relates to a medical device for detecting at least one physiological parameter of a user and to a method of assembling a medical device. The device and the method according to this disclosure may specifically be used for long-term monitoring of an analyte concertation in a body fluid (also referred to herein as a “bodily fluid”), such as for long-term monitoring of a blood glucose level or of the concentration of one or more other types of analytes in a body fluid of the user, specifically a body fluid contained in a body tissue of the user. This disclosure may both be applied in the field of home care as well as in the field of professional care, such as in hospitals. Other applications are feasible.

Monitoring certain body functions, more particularly monitoring one or more physiological parameters of a user, more specifically monitoring one or more concentrations of certain analytes, plays an important role in the prevention and treatment of various diseases. Without restricting further possible applications and further body functions, this disclosure will be described in the following text with reference to blood-glucose monitoring. However, additionally or alternatively this disclosure can also be applied to other types of physiological parameters and/or other types of analytes.

Blood glucose monitoring, besides by using optical measurements, specifically may be per-formed by using electrochemical biosensors. Examples of electrochemical biosensors for measuring glucose, specifically in blood or other body fluids, are known from U.S. Pat. Nos. 5,413,690 A, 5,762,770 A, 5,798,031 A, 6,129,823 A or U.S. Publication No. 2005/0013731A1. These or other electrochemical sensors may also be used as analytical sensors in this disclosure.

In addition to so-called spot measurements, in which a sample of a body fluid is taken from a user in a targeted fashion and examined with respect to the analyte concentration, continuous measurements are increasingly becoming established. Thus, in the recent past, continuous measuring of glucose in the interstitial tissue (also referred to as continuous monitoring, CM), for example, has been established as another important method for managing, monitoring and controlling a diabetes state.

In the process, an active sensor region is applied directly to the measurement site, which is generally arranged in a body tissue of the user, specifically in the interstitial tissue, and, for example, converts glucose into electrical charge, e.g., by using an enzyme, such as glucose oxidase, GOD, which charge is related to the glucose concentration and can be used as a measurement variable. Examples of such transcutaneous measurement systems are described in U.S. Pat. No. 6,360,888B1, in U.S. Publication No. 2008/0242962A1, in EP 3 202 323 A1, or in EP 3 202 324 A1. All of these transcutaneous measurement systems may also be modified along the lines of this disclosure.

Hence, current continuous monitoring systems typically are transcutaneous systems, transdermal systems or subcutaneous systems, wherein these expressions, in the following, will be used equivalently. This means that the actual sensor or at least a measuring portion of the sensor is arranged under the skin of the user. However, an evaluation and control part of the system, also referred to as a patch, is generally situated outside of the body of the user, outside of the human or animal body. In the process, the sensor is generally applied using an insertion element or insertion instrument, which is also described, e.g., in U.S. Pat. No. 6,360,888B 1 , in U.S. Publication No. 2008/0242962 A1, in EP 3 202 323 A1, or in EP 3 202 324 A1 in an exemplary fashion. Other types of medical systems are also known.

The analytical sensor typically comprises a substrate, such as a flat substrate, onto which an electrically conductive pattern of electrodes, conductive traces and contact pads may be applied. In use, the conductive traces typically are electrically insolated by using one or more electrically insulating materials. The electrically insulating material typically further also acts as a protection against humidity and other detrimental substances and, as an example, may comprise one or more cover layers such as resists.

As outlined above, in transcutaneous systems, a control part is typically required, which may be located outside the body tissue and which has to be in communication with the sensor. Typically, this communication is established by providing at least one electrical contact, also referred to as an electrical connection, between the analytical sensor and the control part, specifically an electronics unit of the control part, which may be a permanent electrical contact or a releasable electrical contact. Examples of electrical contacts for contacting a triangular assembly of contact pads are shown, e.g., in DE 954712 B. Other techniques for providing electrical contacts, such as by appropriate spring contacts, are generally known and may be applied.

In order to avoid detrimental effects of the aggressive environment onto the electronics unit and/or onto the conductive properties of the electrical contact, the region of the entire electronics unit and, specifically, the electrical contact, is typically encapsulated and protected against humidity. Generally, encapsulations of electrical locks and contacts by using appropriate seals is known from, e.g., DE 200 20 566 U1. Specifically in transcutaneous or subcutaneous sensors, in which the region of electrical contact between the sensor and the control part is close to the human skin, an efficient protection against humidity, dirt, sweat and detergents, such as detergents used for body care, is crucial. In order to provide sufficient protection, the electronics unit typically is fully or partially received within a housing. The housing, as an example, may be made of at least one plastic material, such as of at least one thermoplastic material. Again, for exemplary embodiments of suitable housings, reference may be made to any one of U.S. Pat. No. 6,360,888B1, U.S. Publication No. 2008/0242962A1, EP 3 202 323 A1, or EP 3 202 324 A1.

In medical devices and medical systems comprising analytical sensors for transcutaneous insertion into a body tissue, the integration of the analytical sensor into the medical device and, specifically, into the housing of the medical device, is technically challenging. Thus, U.S. Pat. No. 10,226,207 B2 and U.S. Publication No. 2011/0190603 Al describe methods, devices and systems for providing a sensor insertion assembly. The sensor insertion assembly includes an inserter housing, an introducer including a body portion having a proximal end and a distal end and a shaft portion comprising a channel and a distal end. The shaft portion extends downwardly from an edge of the body portion, and includes a holding member disposed along a length of the channel. The holding member is configured to substantially releasably retain the analyte. Specifically, a sponge material may be disposed along the channel of the shaft portion, the sponge material being configured to provide a soft interference fit with a sensor disposed in the shaft portion.

Despite the advantages provided by the known devices and methods, several technical challenges remain. Specifically, miniaturization remains an issue. Thus, specifically, miniaturizing the control part, also often referred to as a patch or body patch, is technically challenging. In order to improve convenience and comfort of use, the control part typically is constructed rather flat, in order to provide, in use, a low profile and a low protrusion from the skin of the user. For this purpose, however, the analytical sensors often, outside the body tissue, are positioned such that their substrates are essentially parallel to the body surface. In order to extend into the body tissue, however, the analytical sensors have to be bent, such as by 90°, to protrude from the control part into the body tissue. The bending portion of the analytical sensor, however, is a vulnerable element of the medical device. Specifically, the bending portion typically is located in a region in which the analytical sensor protrudes from the housing of the control part. As an example, the housing may have a small opening through which the sensor may protrude. The housing typically comprises a plurality of housing parts, such as an upper housing part and a lower housing part, wherein the housing parts, as an example, are connected by one or more adhesives. Adhesives, however, may flow, through the opening, into a sensor channel outside the housing.

Consequently, several detrimental influences may act on the analytical sensor, including stress due to the bending of the analytical sensor, which may be increased by the stress exerted by the adhesive. In addition, due to the rigid connection of the analytical sensor to the control part or patch, movements of the insertable portion of the analytical sensor exerted by movements of the body tissue of the user may lead to additional notch stress in the bending region.

The detrimental influences specifically may be considered when the analytical sensor comprises a plurality of layers, as may also be the case in this disclosure. Thus, specifically, the stress of various root causes may lead to a delamination of the layers, may lead to a breaking of one or more of the layers, or may even lead to breakage of the entire analytical sensor. Thus, the detrimental influences may lead to various types of damages in the analytical sensor. As a consequence, faulty measurements or even systemic failures may occur, specifically in medical devices having a high degree of miniaturization.

### SUMMARY

This disclosure teaches a medical system and a method of assembling a medical device, which fully or partially address the above-mentioned technical challenges of known devices and methods of similar kind. Specifically, devices and methods are taught herein which at least partially mitigate the above-mentioned technical influences of the bending of the analytical sensor. More specifically, means and methods are disclosed which improve the guiding of the analytical sensor through the housing of the medical device, even though a rigid housing material may be used and even though the analytical sensor may be bent at the position in which the analytical sensor leaves the housing.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “electronics unit,” “analytical sensor,” “housing” and “guide,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of this disclosure, a medical device for detecting, specifically for measuring, at least one physiological parameter of a user is proposed. The medical device comprises:
at least one electronics unit;
at least one analytical sensor for transdermal insertion into a body tissue of a user, the analytical sensor having an insertable portion configured for at least partially being inserted into the body tissue and at least one electrical connection portion being electrically connected to the electronics unit; and
at least one housing being made of at least one housing material, the housing receiving the electronics unit.

The analytical sensor protrudes from at least one opening in the housing with at least the insertable portion. Thus, at least one part of the at least one insertable portion of the analytical sensor protrudes from the opening and the housing. Further, the medical device comprises at least one guiding element (also referred to herein as a “guide”) made of at least one guiding material softer than the housing material. The guiding element, at the opening and specifically within the opening, at least partially and specifically fully surrounds the analytical sensor.

The term “medical device” as used herein is a broad term and is to be given its ordinary and customary meaning to a person of ordinary skill in the art and is not to be limited to a special or customized meaning. The term specifically may refer, without limitation, to an arbitrary device configured for conducting at least one medical analysis and/or at least one medical procedure. The medical device therefore generally may be an arbitrary device configured for performing at least one diagnostic purpose and/or at least one therapeutic purpose. In the present case, the medical device is configured for detecting the at least one physiological parameter of the user, as will be outlined in further detail below. The medical device specifically may comprise an assembly of two or more components capable of interacting with each other, such as in order to perform one or more diagnostic and/or therapeutic purposes, such as in order to perform the medical analysis and/or the medical procedure. Specifically, the two or more components may be capable of performing at least one detection of the at least one analyte in a body tissue, specifically in a body fluid contained in the body tissue.

As outlined above in the context of the description of known systems of similar kind, the medical device specifically may comprise at least one control part and the at least one analytical sensor electrically connected to the control part. The control part, often also referred to as a “patch,” may electrically control at least one measurement of the medical device by using the analytical sensor. The control part may comprise the electronics unit as will be outlined in further detail below. The control part may fully or partially be located, directly or indirectly with one or more layers of matter interposed, on a body surface of the user, whereas the analytical sensor, with the at least one insertable portion, may transcutaneously protrude into the body tissue of the user.

As generally used within this disclosure, the terms “patient” and “user” may refer to a human being or an animal, independent from the fact that the human being or animal, respectively, may be in a healthy condition or may suffer from one or more diseases. As an example, the patient or the user may be a human being or an animal suffering from diabetes. However, additionally or alternatively, this disclosure may be applied to other types of users or patients or diseases.

The term “detect” as used herein is a broad term and is to be given its ordinary and customary meaning to a person of ordinary skill in the art and is not to be limited to a special or customized meaning. The term specifically may refer, without limitation, to the process of qualitatively and/or quantitatively determining the at least one physiological parameter. Thus, specifically, the medical device may be configured for measuring the at least one physiological parameter. More specifically, the medical device may be configured for detecting at least one analyte in a body fluid of the user, specifically in a body fluid contained in at least one body tissue of the user. Thus, specifically, the detecting may comprise determining the presence and/or the quantity and/or the concentration of the at least one analyte. Thus, the detection may be or may comprise a qualitative detection, simply determining the presence of the at least one analyte or the absence of the at least one analyte, and/or may be or may comprise a quantitative detection, which determines the quantity and/or the concentration of the at least one analyte. As a result of the detection, at least one signal may be produced which characterizes an outcome of the detection, such as at least one measurement signal. The at least one signal specifically may be or may comprise at least one electronic signal such as at least one voltage and/or at least one current. The at least one signal may be or may comprise at least one analogue signal and/or may be or may comprise at least one digital signal. As further used herein, the term “determining a concentration” generally may refer to a process of generating at least one representative result or a plurality of representative results indicating the concentration of the analyte in the body fluid.

The term “physiological parameter” generally, as used herein is a broad term and is to be given its ordinary and customary meaning to a person of ordinary skill in the art and is not to be limited to a special or customized meaning. The term specifically may refer, without limitation, to at least one parameter describing or being representative of at least one state and/or function of the body of the user or of a part thereof. Specifically, the physiological parameter, as outlined above, may be or may comprise the presence and/or the concentration of at least one analyte, such as in at least one body tissue and/or in at least one body fluid of the user. As used herein, the term “analyte” is a broad term and is to be given its ordinary and customary meaning to a person of ordinary skill in the art and is not to be limited to a special or customized meaning. The term specifically may refer, without limitation, to an arbitrary element, component or compound which may be present in the body tissue and/or in the body fluid and the presence and/or the concentration of which may be of interest for the user, the patient or medical staff such as a medical doctor. Particularly, the analyte may be or may comprise an arbitrary chemical substance or chemical compound which may take part in the metabolism of the user or the patient, such as at least one metabolite. As an example, the at least one analyte may be selected from the group consisting of glucose, cholesterol, triglycerides, and/or lactate. Additionally or alternatively, however, other types of analytes may be determined and/or any combination of analytes may be determined. The detection of the at least one analyte specifically may be an analyte-specific detection. As further used herein, the term “body fluid,” also referred to as “body fluid,” generally may refer to a fluid which typically is present in a body or body tissue of the user or the patient and/or which may be produced by the body of the user or the patient. As an example for body tissue, interstitial tissue may be named. Thus, as an example, the body fluid may be selected from the group consisting of blood and interstitial fluid. However, additionally or alternatively, one or more other types of body fluids may be used, such as saliva, tear fluid, urine or other body fluids. During detection of the at least one analyte, the body fluid may be present within the body or body tissue. Thus, specifically, as will be outlined in further detail below, the analytical sensor may be configured for detecting at least one analyte in a body tissue, specifically in a body fluid contained in the body tissue. Thus, specifically, the analytical sensor may be an in vivo analytical sensor.

As outlined above, the medical device comprises the at least one electronics unit. For purposes of this disclosure, the term “electronics unit” is used interchangeably with the term “electronics.” The term “electronics unit,” or just “electronics,” as used herein is a broad term and is to be given its ordinary and customary meaning to a person of ordinary skill in the art and is not to be limited to a special or customized meaning. The term specifically may refer, without limitation, to a component or a combination of components of the medical device comprising at least one electronic component, such as at least one passive and/or at least one active electronic component. The electronics unit, specifically, may comprise at least one integrated circuit. Thus, as an example, the electronics unit may comprise at least one of a semiconductor amplifier circuit and a semiconductor processor circuit. The electronics unit, as also outlined in further detail below, specifically may comprise at least one circuit carrier, specifically at least one circuit board and more specifically at least one printed circuit board, with one or more electronic components, such as one or more active and/or passive electronic components, attached to, integrated into or connected to the circuit carrier. The circuit carrier, as an example, may comprise one or more conductive traces interconnecting different electronic components. The circuit carrier, as an example, may be or may comprise a flat circuit carrier, such as a flat circuit board, which specifically may be oriented parallel to the body surface of the user within the electronics unit. Thus, specifically, the electronics unit may be or may comprise at least one printed circuit board having one or more electronic components attached thereto, such as one or more integrated circuits. Additionally or alternatively, the electronics unit may also comprise at least one energy storage device, such as at least one of a battery, and accumulator or a supercap.

The electronics unit, and optionally the housing surrounding the electronics unit, may form or may form part of a control portion of the medical device. The term “control portion,” also often referred to as a “measurement unit,” as used herein, is a broad term and is to be given its ordinary and customary meaning to a person of ordinary skill in the art and is not to be limited to a special or customized meaning. The term specifically may refer, without limitation, to an arbitrary functional element configured for performing or controlling at least one operation, such as, in the present case, the detecting of the at least one physiological parameter of the user, more specifically the qualitative and/or a quantitative detection of the at least one analyte. The control portion, as an example, may form or may partially be formed as a patch attachable to the body surface of the user. For exemplary embodiments or patches, reference may be made, as an example, to the above-mentioned documents EP 3 202 323 A1 or EP 3 202 324 A1, the electronics units and patches of which may also be modified along the lines of this disclosure.

As further outlined above, the medical device comprises the at least one analytical sensor for transdermal insertion into a body tissue of a user. The analytical sensor comprises the insertable portion configured for at least partially being inserted into the body tissue and the at least one electrical connection portion being electrically connected to the electronics unit. The term “analytical sensor,” as used herein, is a broad term and is to be given its ordinary and customary meaning to a person of ordinary skill in the art and is not to be limited to a special or customized meaning. The term specifically may refer, without limitation, to an arbitrary element which is adapted to qualitatively and/or quantitatively detecting the at least one physiological parameter of the user, specifically to qualitatively and/or quantitatively detecting the at least one analyte in the body tissue of the user, specifically in at least one body fluid contained in the body tissue of the user, and/or which is adapted to be used in the process of detection of the physiological parameter. The analytical sensor, without restricting the option of detecting other types of physiological parameters, may also be referred to as an “analyte sensor,” and the analyte sensor specifically may be adapted to determine the concentration of the analyte and/or a presence of the analyte in the body fluid.

The analytical sensor specifically may be an electrochemical sensor. The term “electrochemical sensor” as used herein is a broad term and is to be given its ordinary and customary meaning to a person of ordinary skill in the art and is not to be limited to a special or customized meaning. The term specifically may refer, without limitation, to a sensor which is configured to conduct an electrochemical measurement in order to detect the at least one physiological parameter, specifically in order to detect the at least one analyte contained in the body fluid. The analytical sensor specifically may have at least two electrodes which may be exposed to the body fluid, such as at least one working electrode and at least one of a counter electrode and a reference electrode. The term “electrochemical measurement” refers to a detection of an electrochemically detectable property of the analyte, such as an electrochemical detection reaction. Thus, for example, the electrochemical detection reaction may be detected by comparing one or more electrode potentials and/or by measuring one or more electrical currents and/or one or more electrical voltages. The electrochemical sensor specifically may be adapted to and/or may be usable to generate at least one electrical sensor signal which directly or indirectly indicates the presence and/or the extent of the electrochemical detection reaction, such as at least one current and/or at least one voltage. The detection may be analyte-specific. The measurement may be a qualitative and/or a quantitative measurement. Still, other embodiments are feasible.

The analytical sensor is configured for transdermal insertion into a body tissue of a user, so the analytical sensor also may be referred to as a “transcutaneous sensor” or as a “transdermal sensor.” The terms “transcutaneous sensor” or “transdermal sensor” as used herein are broad terms and are to be given their ordinary and customary meaning to a person of ordinary skill in the art and are not to be limited to a special or customized meaning. The terms specifically may refer, without limitation, to an arbitrary sensor which is adapted to be at least partly arranged within the body tissue of the patient or the user, with at least one part of the sensor being located outside the body tissue. For the purpose of being inserted into the body tissue, the analytical sensor comprises the insertable portion.

The term “insertable portion” as used herein is a broad term and is to be given its ordinary and customary meaning to a person of ordinary skill in the art and is not to be limited to a special or customized meaning. The term specifically may refer, without limitation, to a part or component of an element configured to be insertable into an arbitrary body tissue.

In order to further render the analytical sensor to be usable as a transcutaneous sensor, the analyte sensor, specifically the insertable portion, may fully or partially provide a biocompatible surface, i.e., a surface which, at least during durations of use, do not have any detrimental effects on the user, the patient or the body tissue. Specifically, the insertable portion of the analyte sensor may have a biocompatible surface, e.g., a biocompatible coating surrounding at least the insertable portion of the analytical sensor. As an example, the analytical sensor, specifically the insertable portion, may fully or partially be covered with at least one biocompatible membrane, such as at least one polymer membrane or gel membrane which is permeable for the at least one analyte and/or the at least one body fluid and which, on the other hand, retains sensor substances such as one or more test chemicals within the sensor and prevents a migration of these substances into the body tissue. Other parts or components of the analyte sensor may stay outside of the body tissue. The other parts may be connectable to an evaluation device such as to the electronics units as will further be described below. The insertable portion, as an example, may have an elongated shape, such as a basically rectangular elongated shape, with a longer side having a length of 5 mm to 30 mm. The shorter side of the rectangle may, as an example, have a width of no more than 5 mm, specifically of no more than 2 mm. The analytical sensor, specifically the insertable portion, may be made of at least one flexible material, specifically of a laminated flexible material such as a foil material. Thus, the insertable portion of the analytical sensor specifically may be flexible and/or deformable. Generally, the analytical sensor may be dimensioned such that a transcutaneous insertion is feasible, such as by providing a width in a direction perpendicular to an insertion direction of no more than 5 mm, preferably of no more than 2 mm, more preferably of no more than 1.5 mm. The sensor may have a length of less than 50 mm, such as a length of 30 mm or less, e.g., a length of 5 mm to 30 mm. As used herein, the term “length” may refer to a direction parallel to the insertion direction. It shall be noted, however, that other dimensions are feasible.

As further outlined above, the analytical sensor further comprises at least one electrical connection portion that is electrically connected to the electronics unit. The term “electrical connection portion” as used herein is a broad term and is to be given its ordinary and customary meaning to a person of ordinary skill in the art and is not to be limited to a special or customized meaning. The term specifically may refer, without limitation, to a portion configured for being electrically connected to another part, element or device. Thus, the electrical connection portion specifically may comprise one or more electrical contact pads, such as contact pads being fully or partially made of at least one electrically conductive material, such as of at least one metal such as gold and/or platinum, and/or of at least one electrically conductive polymer and/or of at least one electrically conductive inorganic material, such as carbon. The contact pads, as an example, may have a round shape, an oval shape, or a polygonal shape, such as a rectangular shape. The contact pads, as an example, may be connected to one or more electrodes of the analytical sensor being located in the insertable portion of the sensor, via one or more electrically conductive traces, such that the one or more electrodes may be electrically contacted via the one or more contact pads. The electrical connection portion may have a shape different to the shape of the insertable portion, such as by providing a higher width, in order to simplify electrical contacting. In general, the insertable portion and the electrical connection portion both may comprise the same substrate, e.g., a flexible substrate, such as a polymer substrate, e.g., a polyimide substrate. Other embodiments, however, are feasible.

The electrical connection portion is electrically connected to the electronics unit. For this purpose, the electronics unit may comprise at least one contact element configured to electrically contact the connection portion of the analytical sensor. The contacting may be performed, as an example, by soldering, wire bonding and/or by other electrically contacting techniques, such as by one or more clamps, zebra connectors or electrical contact pins being pressed onto the electrical connection portion of the analytical sensor. The electrical connection between the electronics unit and the electrical connection portion of the analytical sensor may be embodied such that electronics unit may control at least one sensor operation using the analytical sensor and/or such that the electronics unit may measure at least one electrical parameter provided by the analytical sensor, such as at least one of a current and a voltage. For this purpose, the electronics unit may comprise at least one measurement device, such as at least one voltage measurement device and/or at least one current measurement device.

In addition to the electrical connection portion being electrically connected to the electronics unit, the analytical sensor may also be mechanically connected to one or both of the electronics unit and the housing. Thus, as will be outlined in further detail below, the analytical sensor, specifically within the housing, may also be mechanically fixated to one or both of the electronics unit and the housing by one or more adhesives, such as by gluing the analytical sensor to the electronics unit.

As further outlined above, the medical device comprises at least one housing made of at least one housing material. The housing and the electronics unit may form, as outlined above, a control part of the medical device, such as a patch or body mount or may be parts of a control part. The term “housing” as used herein is a broad term and is to be given its ordinary and customary meaning to a person of ordinary skill in the art and is not to be limited to a special or customized meaning. The term specifically may refer, without limitation, to an arbitrary element or combination of elements which is adapted to fully or partially surround and/or receive one or more other elements in order to provide one or more of a mechanical protection, a mechanical stability, an environmental protection against moisture and/or ambient atmosphere, a shielding against electromagnetic influences or the like, to the one or more other elements surrounded by the housing. Thus, the housing may simply provide a basis for attachment and/or holding one or more further components or elements. Additionally or alternatively, the housing may provide one or more interior spaces for receiving one or more further components or elements. The housing specifically may be a rigid housing which does not macroscopically deform under its own weight. Specifically, the housing may be made of at least one thermoplastic material, as will be outlined in further detail below. The housing specifically be manufactured by at least one molding technique, such as by injection molding. A part of the housing may also be made by using a casting technique, such as a casting technique using an epoxy resin. Specifically, however, the housing may be made of at least two complementary parts which may be assembled to form the housing, the housing specifically having at least one interior space surrounded by the complementary parts, specifically fully surrounded.

As further outlined above, the housing receives the electronics unit. Thus, specifically, the housing may comprise at least one interior space fully or partially surrounded by the housing, wherein the electronics unit is disposed within the interior space. The electronics unit, specifically, may be fixed or fastened within the interior space by the housing, such as by one or more of a form fit, a force fit and a material connection or a substance-to-substance connection. Thus, the electronics unit, as an example, may be clamped to the housing, attached to the housing by one or more screws, glued to the housing or the like.

The housing specifically may have an application side facing the body tissue when the insertable portion of the analytical sensor is inserted into the body tissue. Thus, generally, the term “application side” may refer to a lower side of the housing, specifically a surface of the housing, facing the body tissue, whereas an upper side opposing the application side may face away from the body tissue. As an example, the housing generally may have a flat shape, with a typical lateral extension, e.g., an equivalent diameter, exceeding its thickness by, e.g., at least a factor of two, such as by at least a factor of three or more. As an example, the housing may have a cylindrical shape or the shape of a cube. The application side, as an example, may be essentially flat, e.g., with a circular, an oval, or a polygonal shape, in order to rest on the skin of the user.

As further outlined above, the analytical sensor protrudes from at least one opening in the housing with at least the insertable portion. Thus, at least the insertable portion may fully or partially protrude from the housing, whereas the electrical connection portion may be located within the housing. The at least one opening, as an example, may be formed at an interconnection between at least two housing parts, such as between a lower shell or lower part of the housing and an upper shell or upper part of the housing interconnecting with the lower shell or lower part, wherein the opening is formed, e.g., by a hole or slot at the interconnection between the interconnecting parts. This construction, with the opening being located at the interconnection of at least two housing parts, may simplify assembling of the medical device, since the analytical sensor may be inserted into at least a first one of the housing parts, with the insertable portion protruding through the opening, before at least one second part of the housing parts is connected to the first housing part.

Further, as outlined above, the medical device comprises at least one guiding element or guide made of at least one guiding material softer than the housing material. The guiding element, at the opening and specifically within the opening, at least partially and specifically fully surrounds the analytical sensor. The term “guiding element,” as used herein, is a broad term and is to be given its ordinary and customary meaning to a person of ordinary skill in the art and is not to be limited to a special or customized meaning. The term specifically may refer, without limitation, to an arbitrary element or combination of elements configured and capable of protecting and/or guiding another element. The guiding element, specifically, may be embodied separate from the analytical sensor, such that, during assembly, the guiding element and the analytical sensor are inserted as separate parts. Specifically, the guiding element may partially or preferably fully surround the analytical sensor within the opening. For this purpose, the analytical sensor specifically may be embedded in the at least one guiding element, such that guiding element protects the analytical sensor from at least one, more specifically from at least two and more specifically from all sides, at least in one cross-sectional plane through the opening of the housing.

As also outlined above, the guiding element is at least partially made of at least one guiding material softer than the housing material. For quantifying the partners of the materials, appropriate hardness units may be used, such as Shore A or Shore D.

As outlined above, the guiding element may fully or partially surround the analytical sensor, such that, as an example, within the opening, the analytical sensor is surrounded by guiding element. Generally, for this purpose, the guiding element may be designed in various ways, such as in a way selected from the group consisting of:
the guiding element is separate from the housing and optionally also from the analytical sensor, such as inserted into the housing as a separate part or insert;
the guiding element is fully or partially integrated into the analytical sensor;
the guiding element is fully or partially integrated into the housing, with the housing being a multicomponent housing comprising at least the housing material and the guiding material.

In case the guiding element is fully or partially integrated into the analytical sensor, the analytical sensor may comprise at least one coating comprising the guiding material, specifically at least one circumferential coating. Thus, the analytical sensor and the guiding element may be provided as one single piece. The at least one coating comprising the guiding material may be deposited on at least one surface of the analytical sensor, specifically via at least one method selected from the group consisting of: spray coating, dip coating. However, also other kinds of methods may be possible.

For the latter option, as an example, multicomponent injection molding or insert molding may be used, such that, in at least one housing part of the housing, an insert his molded by multicomponent molding or insert molding, the insert being softer than the surrounding material.

Additionally or alternatively, however, the guiding element may also be designed as an insert into the housing, such as loosely inserted into the housing. Thus, specifically, the insert may be formed as a separate part being separate from the housing and from the analytical sensor which, during assembly of the medical device, may be handled individually from the housing.

The insert specifically may comprise at least one of: a tube partially surrounding the analytical sensor; a sandwich comprising at least one upper insert and at least one lower insert, with the analytical sensor embedded between the upper insert and the lower insert; a foldable insert having at least one insert base component and at least one foldable insert component, with the foldable insert component being foldable over the analytical sensor such that, in a folded state, the analytical sensor is embedded between the insert base component and the foldable insert component. Exemplary embodiments of these types of inserts which may fully or partially surround the analytical sensor, such as by sandwich embedding, will be given further detail below.

The guiding element specifically may comprise at least one curved surface. The analytical sensor may be guided over the curved surface. Thus, generally, the analytical sensor may be in direct physical contact with the guiding element, such that the curvature of the curved surface of the guiding element is transferred onto the analytical sensor in the region of contact between these parts.

More specifically, as outlined above, the analytical sensor may be embedded between at least two guiding elements and/or between at least two parts of one guiding element. Both of these at least two parts may have complementary curved surfaces, wherein the curvature of the curved surfaces may be transferred onto the analytical sensor in the region of contact between the curved surfaces and the analytical sensor. Thus, generally, the guiding element may comprise at least two opposing curved surfaces, wherein the analytical sensor may be guided in between the two opposing curved surfaces.

The housing may exert a force onto the guiding element. Thus, as an example, the guiding element, at the opening of the housing, may be compressed by the housing, specifically by at least one first housing component and at least one second housing component, more specifically by at least one lower housing part and at least one upper housing part, more specifically by at least one lower housing part and at least one of an upper shell of the housing and a through hole part of the housing. This compression, as an example, may lead to a sealing of the opening, wherein a circumferential space between the analytical sensor and a rim of the housing at the opening is filled by the compressed guiding element. The compression may take place when the housing parts are assembled, e.g., by attaching at least one of the housing parts to at least another one of the housing parts by one or more of a force fit connection, a form fit connection or a connection by material connection or substance-to-substance connection.

As outlined above, the hardness of the housing material and the hardness of the guiding material may be compared by appropriate hardness units. Generally, as an example, the housing material may, as will be outlined in further detail below, be fully or partially formed by at least one thermoplastic material, whereas the guiding material, as an example, may be formed by one or more of a soft thermoplastic material, an elastomeric material and a sponge or foam material, such as a polyurethane foam.

As an example, the housing material may have a Shore D hardness of at least 40, specifically a Shore D hardness of 50 to 80. The guiding material specifically may have a Shore A hardness of 0 to 70, specifically a Shore A hardness of 10 to 50. Specifically, the housing material may have a Shore D hardness of at least 40, specifically a Shore D hardness of 50 to 80, and the guiding material may have a Shore A hardness of 0 to 70, specifically a Shore A hardness of 10 to 50. Specifically, the housing material may have a Shore D hardness of 80 and the guiding material may have a Shore A hardness of 0 to 70.

As outlined above, the housing material specifically may comprise at least one thermoplastic material. More specifically, the housing material may be a thermoplastic material which may be selected from the group consisting of: polycarbonate (PC), exemplarily Makrolon 2458; acrylonitrile butadiene styrene copolymer (ABS). However, also other materials may be possible.

As also outlined above, the guiding material specifically may comprise at least one elastomeric material. More specifically, the guiding material may be selected from the group consisting of: a thermoplastic elastomer (TPE), exemplarily THERMOLAST® M TM6ADT; a silicone rubber (VMQ), exemplarily liquid silicone rubber (LSR). However, also other materials may be possible.

The analytical sensor specifically may be bent, more specifically curved. Thus, the analytical sensor may fully or partially be made of a flexible material, such as by using a flexible substrate, as outlined above. More specifically, the bending or curvature may make use of the flexibility of this flexible substrate, by bending the flexible substrate with an excess of curvature being essentially parallel to a surface of the substrate, e.g., with a deviation from a parallel orientation of no more than 20°, specifically of no more than 10°.

More specifically, the analytical sensor may comprise at least one portion having an elongated shape. The insertable portion may be formed by this portion having the elongated shape, or the portion of the elongated shape may comprise the insertable portion. The portion having the elongated shape may fully or partially extend through the opening in the housing. The housing, as outlined above, generally may have an application side facing the body tissue when the insertable portion of the analytical sensor is inserted into the body tissue. Within the housing, the portion having an elongated shape may extend essentially parallel to the application side of the housing, wherein, outside the housing, the portion having the elongated shape may extend in a direction oblique to the application side of the housing, specifically essentially perpendicular to the application side of the housing, e.g., with a deviation from a perpendicular orientation by no more than 30°, specifically by no more than 20°. Thus, in between the portions of the elongated shape being located inside and outside the housing, the curvature of the analytical sensor may be located, wherein the curvature may fully or partially be surrounded by the guiding element. Thus, generally, the guiding element at least partially may surround a bent portion of the analytical sensor, also referred to as the curvature of the analytical sensor.

As outlined above, the curvature specifically may have an axis of curvature essentially perpendicular parallel to a plane of extension of the analytical sensor. Thus, the analytical sensor specifically may have a flat substrate, such as a flat and elongated substrate, wherein an axis of bending of the analytical sensor, also referred to as an axis of curvature, may be oriented essentially parallel to the flat substrate, e.g., with a deviation from a parallel orientation of no more than 20°, specifically of no more than 10°.

As outlined above, the housing may have an application side facing the body tissue when the insertable portion of the analytical sensor is inserted into the body tissue. The housing, on the application side, may comprise at least one adhesive for attachment to the body surface. The adhesive, as an example, may be attached directly to the application side of the housing. Additionally or alternatively, however, the housing may also be attached to an adhesive plaster configured for attachment of the housing to the body surface. The adhesive, generally, may be protected by at least one detachable liner before use.

As also outlined above, the electronics unit specifically may comprise at least one circuit carrier. More specifically, the electronics unit may comprise at least one printed circuit board, with the electrical connection portion being electrically connected to the printed circuit board. Thus, as an example, the printed circuit board may comprise at least one contact pad, with the electrical connection portion of the analytical sensor being directly or indirectly electrically connected to the contact pad. The connection, as outlined above, specifically may be a connection selected from the group consisting of wire bonding, contacting by electrical contact springs or pins, an electrical connection by clamping, an electrical connection by a press-fit using at least one conductive element, or the like.

As also indicated above, the housing specifically may comprise at least one interior space. The interior space, as an example, may be formed by two or more housing parts. The electronics unit may be embedded in the interior space. The embedding, as an example, may imply a fixing of the electronics unit in the interior space, such as by one or more of a force fit connection, a form fit connection and a substance-to-substance connection or material connection. The interior space, specifically, may have a round shape, such as a cylindrical shape or a toroidal shape.

Specifically, the housing may comprise at least one through hole for receiving an insertion element during insertion of the insertable portion of the analytical sensor. As an example, the through hole may be placed centrally in the housing, with the housing surrounding the through hole, e.g., with a rotational symmetry. Other options, however, are feasible. The term “insertion element,” as used herein, is a broad term and is to be given its ordinary and customary meaning to a person of ordinary skill in the art and is not to be limited to a special or customized meaning. The term specifically may refer, without limitation, to an arbitrary element configured for penetrating the skin and the body tissue of the user. For this purpose, the insertion element specifically may comprise at least one of a sharp and a point. More specifically, the insertion element may comprise at least one of a needle and a cannula. The insertion element may comprise at least one receptacle for receiving at least the insertable portion of the analytical sensor during insertion, such as a slot. Thus, during insertion, when moving into the body tissue, the receptacle may receive the insertable portion of the analytical sensor and may transport the insertable portion of the analytical sensor into the body tissue. When retracting the insertion element from the body tissue, the insertable portion of the analytical sensor may remain, at least partially, in the body tissue, whereas the insertion element is removed from the body tissue. During this insertion process, including the penetration of the skin of the user, the transportation of the insertable portion of the analytical sensor into the body tissue and the retraction of the insertion element from the body tissue, the electrical connection portion of the analytical sensor remains electrically connected to the electronics unit which may be placed outside the body tissue, e.g., with a housing and the electronics unit and/or the control part being placed directly or indirectly on the skin of the user. Thus, the insertable element may be movable with respect to the housing of the medical device. The insertion element may be driven manually and/or may be driven by an appropriate actuator which also may be part of the medical device. For exemplary embodiments of insertion processes and/or actuators, reference may be made to any one of U.S. Pat. No. 6,360,888B1 , U.S. Publication No. 2008/0242962A1 , EP 3 202 323 A1, or EP 3 202 324 A1.

The through hole specifically may provide space and/or mechanical guidance for the insertion element. Thus, as outlined above, the housing may have an application side facing the body tissue and opposing upper side. During insertion, the insertion element may fully or partially be placed in the through hole. After insertion, the insertion element may be removed from the through hole. Thus, in a functional state during which the at least one physiological parameter is detected, the insertion element may be removed from the medical device.

During insertion, the insertion element may extend from an upper side of the housing, the opposite side opposing the application side of the housing, to the application side, through the through hole.

As outlined above, the through hole specifically may be surrounded by the housing. The opening in the housing, through which the analytical sensor protrudes, may lead into the through hole. Thus, specifically, the opening in the housing may extend or lead into the through hole, and the analytical sensor or at least part of the insertable portion of the analytical sensor may protrude, from the housing, into the through hole, through the opening. The above-mentioned curvature of the analytical sensor may be located at least partially within the through hole, so the analytical sensor may then extend, via the through hole, to the application side of the housing and protrude therefrom, and may protrude, in the inserted state, into the body tissue.

The housing specifically may comprise at least one through hole part surrounding the through hole. Thus, as an example, the housing may comprise a part having a ring-shaped opening which surrounds at least a part of the through hole in the housing. The through hole part specifically may be part of an upper housing part which, together with a lower housing part or base plate of the housing, forms a closed housing.

As outlined above, the housing may comprise at least one upper housing part and at least one lower housing part. The lower housing part, as an example, may also be embodied as or referred to as a base plate or a base component and/or may comprise a base plate or base component. As an example, these housing parts may be formed as a shell and may be joined, during assembly, along at least one connection rim, e.g., by one or more of a form fit connection, a force fit connection and a substance-to-substance connection or material connection, e.g., by gluing. The above-mentioned at least one optional through hole part of the housing, as an example, may be part of the upper housing part and/or of the lower housing part.

The analytical sensor, within the housing, may at least partially be fixated by at least one adhesive, such as at least one glue and/or at least one epoxy resin. Thereby, the analytical sensor may be stabilized against movements, and a transfer of movements of the patient's or user's body tissue, via the analytical sensor, onto the connecting portion may be reduced, thereby increasing the stability of the electrical connection.

As further outlined above, the opening of the housing may be sealed by the guiding element. Thus, the opening may be limited by an opening rim, specifically an opening rim of the housing, the opening rim surrounding the opening. The analytical sensor may be surrounded by the opening rim when protruding through the opening. In between the analytical sensor and the opening rim, a circumferential space fully or partially surrounding the analytical sensor may remain. Thus circumferential space may at least partially and specifically fully be filled by the guiding element. As outlined above, the guiding element specifically may seal this circumferential space, in order to prevent water and/or adhesive from passing through the opening. As further outlined above, the guiding element may be compressed by the housing, which may intensify the sealing effect of the guiding element in the opening.

In a further aspect of this disclosure, a medical system is proposed. The term “system” as used herein is a broad term and is to be given its ordinary and customary meaning to a person of ordinary skill in the art and is not to be limited to a special or customized meaning. The term specifically may refer, without limitation, to an arbitrary set of interacting or interdependent components parts forming a whole. Specifically, the components may interact with each other in order to fulfill at least one common function. The at least two components may be handled independently or may be coupled or connectable. The term “medical system,” as used herein, thus, is a broad term and is to be given its ordinary and customary meaning to a person of ordinary skill in the art and is not to be limited to a special or customized meaning. The term specifically may refer, without limitation, to a system configured for performing at least one medical function, specifically a medical function selected from the group consisting of a diagnostic and a therapeutic function. Specifically, the medical system is configured for detecting the at least one physiological parameter of the user, as outlined above. More specifically, as also outlined above, the medical system may be configured for qualitatively and/or quantitatively detecting at least one analyte in a body fluid, such as in a body fluid contained in a body tissue of a user. The medical system specifically may be configured for performing at least two actions, which are the action of inserting the analytical sensor or at least a part thereof, such as the insertable portion, into the body tissue and to the action of detecting the analyte in the body fluid by using the analytical sensor. The medical system specifically may be, in a basic state before use, a unitary system which may be handled as one single piece. After use, which is after insertion of the analytical sensor into the body tissue, the medical system may disassemble into a disposable handling component including an insertion element or inserter in a used state, and into an analytical sensor unit, with a control part or body mount and the analytical sensor, wherein the body mount may be attached to the skin of the user and wherein the analytical sensor may protrude from the analytical sensor unit into the body tissue.

The medical system as proposed herein comprises the medical device as proposed herein, such as according to any one of the embodiments described above and/or according to any one of the embodiments described in further detail below. Further, the medical system comprises at least one insertion element, as discussed above, such as at least one insertion needle and/or at least one insertion cannula. The insertable portion of the analytical sensor protruding from the opening and the housing of the medical device, before insertion, is at least partially received in the insertion element. Thus, as outlined above, the insertion element may comprise at least one receptacle for receiving the analytical sensor or a part thereof, such as insertable portion of the analytical sensor or a part thereof. The receptacle, as an example, may be or may comprise a slot, with the insertable portion of the analytical sensor being at least partially received in the slot before insertion. During insertion, the insertion element may transport the insertable portion or a part thereof into the body tissue in a forward movement. Afterwards, in a rearward movement, insertion element may be retracted from the body tissue, whereas the insertable portion or at least a part thereof remains in the body tissue. The insertion may be driven, as outlined above, manually and/or by using at least one actuator, which may also form part of the medical system.

In a further aspect of this disclosure, a method of assembling a medical device is disclosed. The medical device is configured for detecting at least one physiological parameter of a user. The method comprises the following steps which specifically may be performed in the given order. However, a different order is also feasible. Further, it is possible to perform two or more or even all of the method steps in a fashion overlapping in time or at least partially simultaneously. It is further possible to perform each of the method steps only once or repeatedly. The method may comprise additional method steps which are not listed herein.

The method comprises the following steps:
a. providing at least one electronics unit;
b. providing at least one analytical sensor for transdermal insertion into a body tissue of a user, the analytical sensor having an insertable portion configured for at least partially being inserted into the body tissue and at least one electrical connection portion;
c. electrically connecting the electrical connection portion of the analytical sensor to the electronics unit;
d. providing at least one housing being made of at least one housing material;
e. providing at least one guiding element, the guiding element at least partially being made of at least one guiding material softer than the housing material; and
f. receiving the electronics unit in the housing, such that the analytical sensor, with at least the insertable portion, protrudes from at least one opening in the housing, wherein the guiding element at least partially surrounds the analytical sensor at the opening.

For further definitions and options, reference may be made to the description of the medical device and the medical system as given herein. Thus, specifically, the medical device as assembled by the method may be a medical device according to this disclosure, such as according to any one of the embodiments described above and/or according to any one of the embodiments described in further detail below.

The medical device, the medical system and the method of assembling the medical device provide a large number of advantages over known devices, systems and methods of similar kind and purpose. Thus, specifically, the above-mentioned technical challenges of known systems and methods are and efficiently addressed. In many medical devices for detecting at least one physiological parameter, as well as also in the present case, the analytical sensor may at least partially be fixated by using one or more adhesives, such as glue and/or epoxy resin, which partially surrounds the analytical sensor within the housing. The glue, however, may contribute to stress effects exerting stress onto the sensor, specifically in the opening of the housing. By using the soft guiding element, the technical stress may be reduced. Thus, the guiding element specifically may comprise a soft elastic material component as the guiding material or as a part thereof. The guiding element may provide guiding and protection function. The guiding element, as an example, may be mounted on the base plate of the housing and on the upper sensor fixation element. Thereby, the analytical sensor, specifically a sensor for long-term measurements or continuous glucose monitoring, may be protected and guided through the housing. Further, mechanical stress, bending forces and friction of the analytical sensor may be reduced and sensor damages may be avoided which may result in a dysfunctionality of the medical device. A flexible and tight sensor guidance may be provided, which specifically may avoid sensor movement which may lead to damages or even breakage of the analytical sensor and, thereby, to a dysfunctionality of the medical device.

The proposed solution also efficiently addresses the above-mentioned technical challenge of miniaturization. Thus, specifically, the control part of a sensor patch may be embodied in a rather flat fashion, increasing wearing comfort on the body surface. The flat construction, generally, may require a sharp bending of the analytical sensor, e.g., by approximately 90°, in order to introduce the analytical sensor into the body tissue and, after insertion, have the analytical sensor protrude basically perpendicularly into the body tissue of the user, through the skin. In present systems, however, the analytical sensor is typically guided over part and rigid plastic parts, through a small opening in the housing. The small opening is typically dimensioned such that the housing parts of the housing do not exert excessive forces onto the analytical sensor. For fixating the sensor and/or for sealing the housing, in this disclosure, an adhesive may be used, such as a glue and/or an epoxy. By the guiding element, it may be prevented that the adhesive flows, through the opening, to an outer region outside the housing, e.g., into the through hole, which may also be referred to as a guiding channel.

By using the guiding element, mechanical stress and tensions exerted onto the analytical sensor, induced by the bending and incremented by the adhesive, may be reduced. Further, the notch stress or notch tension exerted onto the analytical sensor at the opening may be reduced, so the effect of movements of the body tissue and the transfer of stress exerted by these movements may be reduced. Thereby, the risk of breaking of the analytical sensor or of parts thereof or of delamination may also be reduced.

By using the guiding element, the contact and the guidance of the analytical sensor by the hard housing material may be replaced by the guiding element and the guiding material. The guiding element may take over the function of guiding and contacting the analytical sensor. Thereby, the issue of conflicting goals may be addressed: The housing material typically has to be chosen to provide optimum rigidity and protection against detrimental influences such as mechanical shocks. These properties, however, are often detrimental to the sensor guiding and are not suited to reduce a chemical stress onto the analytical sensor. The guiding material, however, may be chosen to provide an optimum and soft guiding of the analytical sensor, reducing stress and reducing the risk of sensor breaking. Further, the guiding material may provide a better sealing effect than typical hard housing materials.

The soft guiding element may, as compared to the hard housing material, be designed in an oversize as compared to the width of the opening. Thus, a cross-section of the guiding element, with the analytical sensor embedded therein, may exceed a cross-section of the opening, such that, during assembly, the guiding element, with the analytical sensor embedded therein, is comprised in the opening. Thereby, leakages may be reduced, and a leakage of glue from an interior space of the housing, through the opening, onto the part of the analytical sensor protruding from the housing may be prevented efficiently. Further, the guiding element, with its soft guiding material, may reduce mechanical stress, notch tensions and, thereby, failures of the medical device.

As outlined above, this disclosure may be embodied in various fashions in the medical device. As an example, the analytical sensor may be implemented into the base plate of the housing, with a base part of the guiding element underneath, the base part of the guiding element being inserted into the base plate or being integrated into the base plate. The analytical sensor may then be covered, from an upper side, with a sensor fixation which may also comprise a part of the guiding element, such as an upper part of the guiding element. Thereby, the analytical sensor, at least in the region of the opening and/or in the region of the curvature, may be surrounded by the soft guiding material, may be sealed and may be guided along the curvature.

Additionally or alternatively, as outlined above, the guiding element may be design, fully or partially, as a foldable guiding element. Thus, as an example, the guiding element may be inserted or integrated into a housing component, such as into the base plate. The analytical sensor may be located, at least in part, onto the guiding element, and, subsequently, the guiding element may be folded over the analytical sensor, thereby surrounding the analytical sensor from both sides.

Further, the guiding element may also be embodied, fully or partially, as a loose guiding element comprising one part or a plurality of parts. As an example, a first part of the guiding element may be inserted in between a first housing part, such as the base plate, and the analytical sensor. The analytical sensor may be placed onto this first guiding element. Further, a second guiding element may be placed on top of the analytical sensor, e.g., between the analytical sensor and a second part of the housing, e.g., between the analytical sensor and a sensor fixation part of the housing, followed by placing the second part of the housing on top of the second guiding element.

Again, additionally or alternatively, the analytical sensor or at least a part thereof may be surrounded by the guiding element before inserting the analytical sensor into the housing. As an example, the guiding element may have a tubular shape and may be placed over the analytical sensor or at least a part thereof, e.g., a part of the insertable portion which is to be located in the opening of the housing. In this embodiment or in other embodiments, the opening may be dimensioned in an oversized fashion, exceeding a lateral extension of the analytical sensor.

Summarizing and without excluding further possible embodiments, the following embodiments may be envisaged:

Embodiment 1: A medical device for detecting at least one physiological parameter of a user, the medical device comprising:
at least one electronics unit;
at least one analytical sensor for transdermal insertion into a body tissue of a user, the analytical sensor having an insertable portion configured for at least partially being inserted into the body tissue and at least one electrical connection portion being electrically connected to the electronics unit; and
at least one housing being made of at least one housing material, the housing receiving the electronics unit,
wherein the analytical sensor, with at least the insertable portion, protrudes from at least one opening in the housing,
wherein the medical device further comprises at least one guiding element, the guiding element at least partially being made of at least one guiding material softer than the housing material, the guiding element at least partially surrounding the analytical sensor at the opening.

Embodiment 2: The medical device according to the preceding embodiment, wherein the guiding element is designed in a way selected from the group consisting of:
the guiding element is separate from the housing;
the guiding element is fully or partially integrated into the housing, with the housing being a multicomponent housing comprising at least the housing material and the guiding material.

Embodiment 3: The medical device according to any one of the preceding embodiments, wherein the guiding element is inserted into the housing as at least one insert.

Embodiment 4: The medical device according to the preceding embodiment, wherein the insert comprises at least one of: a tube partially surrounding the analytical sensor; a sandwich comprising at least one upper insert and at least one lower insert, with the analytical sensor embedded between the upper insert and the lower insert; a foldable insert having at least one insert base component and at least one foldable insert component, with the foldable insert component being foldable over the analytical sensor such that, in a folded state, the analytical sensor is embedded between the insert base component and the foldable insert component.

Embodiment 5: The medical device according to any one of the preceding embodiments, wherein the guiding element comprises at least one curved surface, wherein the analytical sensor is guided over the curved surface.

Embodiment 6: The medical device according to the preceding embodiment, wherein the guiding element comprises at least two opposing curved surfaces, wherein the analytical sensor is guided in between the two opposing curved surfaces.

Embodiment 7: The medical device according to any one of the preceding embodiments, wherein the guiding element, at the opening of the housing, is compressed by the housing, specifically by at least one first housing component and at least one second housing component, more specifically by at least one lower housing part and at least one upper housing part, more specifically by at least one lower housing part and at least one of an upper shell of the housing and a through hole part of the housing.

Embodiment 8: The medical device according to any one of the preceding embodiments, wherein the housing material has a Shore D hardness of at least 40, specifically a Shore D hardness of 50 to 80.

Embodiment 9: The medical device according to any one of the preceding embodiments, wherein the guiding material has a Shore A hardness of 0 to 70, specifically a Shore A hardness of 10 to 50.

Embodiment 10: The medical device according to any one of the preceding embodiments, wherein the housing material has a Shore D hardness of 80 and the guiding material has a Shore A hardness of 20 to 50.

Embodiment 11: The medical device according to any one of the preceding embodiments, wherein the housing material comprises at least one thermoplastic material.

Embodiment 12: The medical device according to any one of the preceding embodiments, wherein the housing material is a thermoplastic material which is selected from the group consisting of: polycarbonate (PC), exemplarily Makrolon 2458; acrylonitrile butadiene styrene copolymer (ABS).

Embodiment 13: The medical device according to any one of the preceding embodiments, wherein the guiding material comprises at least one elastomeric material.

Embodiment 14: The medical device according to any one of the preceding embodiments, wherein the guiding material is selected from the group consisting of: a thermoplastic elastomer (TPE), exemplarily THERMOLAST® M TM6ADT; a silicone rubber (VMQ), exemplarily liquid silicone rubber (LSR).

Embodiment 15: The medical device according to any one of the preceding embodiments, wherein the analytical sensor is bent.

Embodiment 16: The medical device according to the preceding embodiment, wherein the analytical sensor comprises at least one portion having an elongated shape, wherein the portion having the elongated shape extends through the opening in the housing, wherein the housing has an application side facing the body tissue when the insertable portion of the analytical sensor is inserted into the body tissue, wherein, within the housing, the portion having an elongated shape extends essentially parallel to the application side of the housing, wherein, outside the housing, the portion having the elongated shape extends in a direction oblique to the application side of the housing, specifically essentially perpendicular to the application side of the housing.

Embodiment 17: The medical device according to any one of the two preceding embodiments, wherein the guiding element at least partially surrounds a bent portion of the analytical sensor.

Embodiment 18: The medical device according to any one of the three preceding embodiments, wherein the analytical sensor has a flat substrate, wherein an axis of bending of the analytical sensor is essentially parallel to the flat substrate.

Embodiment 19: The medical device according to any one of the preceding embodiments, wherein the housing has an application side facing the body tissue when the insertable portion of the analytical sensor is inserted into the body tissue, wherein the housing, on the application side, comprises at least one adhesive for attachment to the body surface.

Embodiment 20: The medical device according to the preceding embodiment, wherein the electronics unit comprises a printed circuit board, with the electrical connection portion being electrically connected to the printed circuit board.

Embodiment 21: The medical device according to the preceding embodiment, wherein the printed circuit board comprises at least one contact pad with the electrical connection portion being electrically connected to the contact pad.

Embodiment 22: The medical device according to any one of the preceding embodiments, wherein the housing comprises at least one interior space, with the electronics unit being embedded in the interior space.

Embodiment 23: The medical device according to any one of the preceding embodiments, wherein the housing comprises at least one through hole for receiving an insertion element during insertion of the insertable portion of the analytical sensor, wherein the housing has an application side facing the body tissue when the insertable portion of the analytical sensor is inserted into the body tissue, wherein, during insertion, the insertion element extends from an upper side opposing the application side to the application side, through the through hole.

Embodiment 24: The medical device according to the preceding embodiment, wherein the through hole is surrounded by the housing.

Embodiment 25: The medical device according to any one of the two preceding embodiments, wherein the opening in the housing leads into the through hole, wherein the analytical sensor protrudes, from the housing, into the through hole.

Embodiment 26: The medical device according to any one of the three preceding embodiments, wherein the housing comprises at least one through hole part surrounding the through hole.

Embodiment 27: The medical device according to any one of the preceding embodiments, wherein the housing comprises at least one upper housing part and at least one lower housing part.

Embodiment 28: The medical device according to any one of the preceding embodiments, wherein the opening of the housing is sealed by the guiding element.

Embodiment 29: The medical device according to any one of the preceding embodiments, wherein the opening is limited by an opening rim of the housing, wherein the analytical sensor is surrounded by the opening rim, wherein a circumferential space between the analytical sensor and the opening rim is at least partially, specifically fully, filled by the guiding element.

Embodiment 30: A medical system, comprising the medical device according to any one of the preceding embodiments, further comprising at least one insertion element, with the insertable portion protruding from the opening in the housing of the medical device being at least partially received in the insertion element.

Embodiment 31: A method of assembling a medical device for detecting at least one physiological parameter of a user, the method comprising:
a. providing at least one electronics unit;
b. providing at least one analytical sensor for transdermal insertion into a body tissue of a user, the analytical sensor having an insertable portion configured for at least partially being inserted into the body tissue and at least one electrical connection portion;
c. electrically connecting the electrical connection portion of the analytical sensor to the electronics unit;
d. providing at least one housing being made of at least one housing material;
e. providing at least one guiding element, the guiding element at least partially being made of at least one guiding material softer than the housing material; and
f. receiving the electronics unit in the housing, such that the analytical sensor, with at least the insertable portion, protrudes from at least one opening in the housing, wherein the guiding element at least partially surrounds the analytical sensor at the opening.
Embodiment 32: The method according to the preceding embodiment, wherein the medical device is a medical device according to any one of the preceding embodiments referring to a medical device.

### BRIEF DESCRIPTION OF THE DRAWINGS

Further optional features and embodiments will be disclosed in more detail in the subsequent description of embodiments. Therein, the respective optional features may be realized in an isolated fashion as well as in any arbitrary feasible combination, as the skilled person will realize. The scope of this disclosure is not restricted by the preferred embodiments. The embodiments are schematically depicted in the Figures. Therein, identical reference numbers in these Figures refer to identical or functionally comparable elements.

The above-mentioned aspects of exemplary embodiments will become more apparent and will be better understood by reference to the following description of the embodiments taken in conjunction with the accompanying drawings, wherein:

FIG. 1 shows an embodiment of a medical system with a medical device and insertion element in a cross-sectional view;

FIG. 2 shows a first embodiment of a guiding element for implementation into the medical device of FIG. 1;

FIG. 3 shows a second embodiment of a guiding element for implementation into the medical device of FIG. 1;

FIG. 4 shows a third embodiment of a guiding element for implementation into the medical device of FIG. 1;

FIGS. 5A and 5B show details of a fourth embodiment of a guiding element for implementation into the medical device of FIG. 1; and

FIG. 6 shows a flow chart of an embodiment of a method of assembling a medical device.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

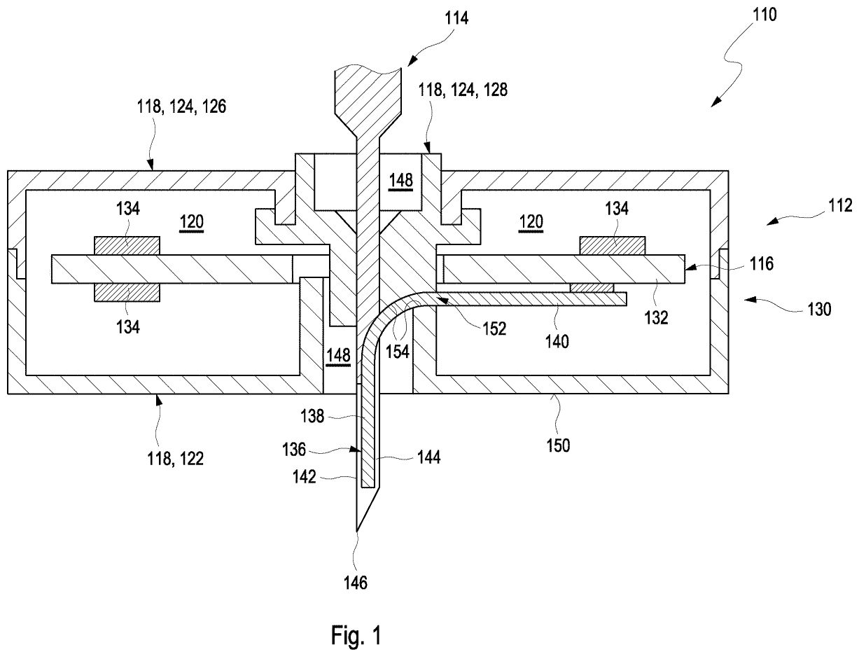

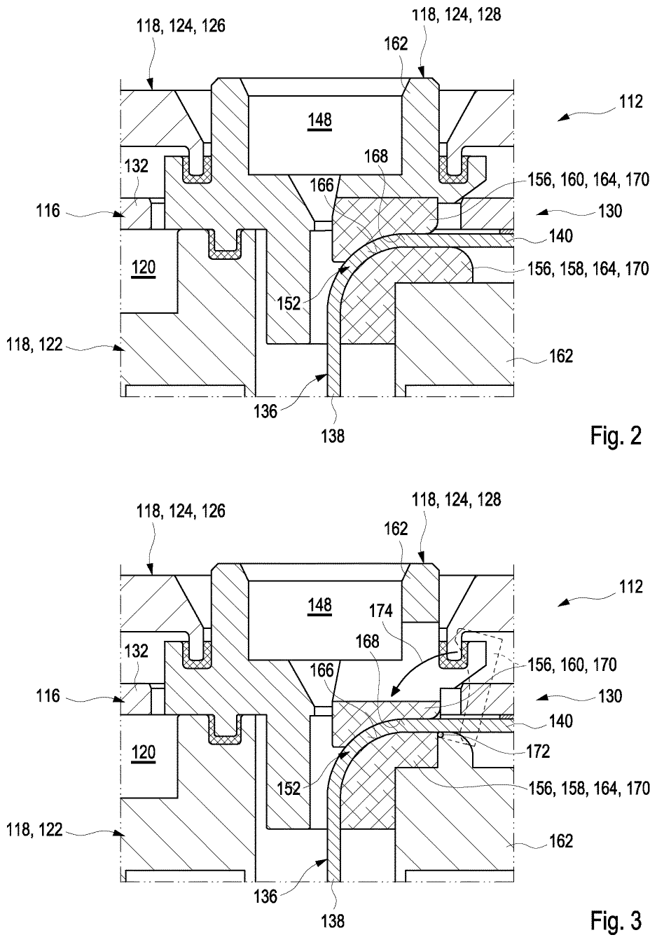

In FIG. 1, a cross-sectional view of an exemplary embodiment of a medical system 110 is shown. The medical system 110 is configured for detecting at least one physiological parameter of a user. For this purpose, the medical system 110 comprises a medical device 112 for detecting the at least one physiological parameter of the user, and, further, at least one insertion element 114. In FIGS. 2, 3, 4 and 5A and 5B, details of four different embodiments of the medical device 112 are shown, which may be implemented in the embodiment shown in FIG. 1. Thus, FIG. 1 and FIGS. 2, 3, 4 and 5A and 5B, in the following, will be discussed in conjunction.

The medical device 112 comprises an electronics unit 116 and a housing 118 having an interior space 120 receiving the electronics unit 116. In the exemplary embodiment shown in FIG. 1, as an example, the housing comprises a plurality of housing parts, such as a lower housing part 122, also referred to as a base plate or a lower shell, and at least one upper housing part 124 which, in this embodiment or in other embodiments, specifically may comprise an upper shell 126 and, additionally, a through hole part 128, which may also be referred to as a sensor fixation or as a needle guiding element, irrespective of the type of the insertion element 114 actually used. The electronics unit 116 and the housing 118, in conjunction, may form a control part 130 of the medical device 112. The electronics unit 116, as an example, may comprise at least one circuit carrier 132 and a plurality of electronic components 134 attached thereto or integrated therein, such as one or more of active electronic components, specifically integrated circuits, passive electronic components and energy storage devices.

The medical device 112 further comprises at least one analytical sensor 136. The analytical sensor 136 is configured for transdermal insertion into a body tissue of the user. For this purpose, as shown in FIG. 1, the analytical sensor 136 comprises an insertable portion 138 configured for at least partially being inserted into the body tissue of the user. Further, the analytical sensor 136 comprises at least one electrical connection portion 140 being electrically connected to the electronics unit 116 within the housing 118.

As shown in the exemplary embodiment of FIG. 1, the insertion element 114 specifically may be embodied as or may comprise a cannula 142. The insertion element 114 may comprise a receptacle 144, such as a slot, in which the insertable portion 138 or at least a part thereof is disposed during insertion into the body tissue. During insertion, a sharp 146 or point of the insertion element 114 penetrates the skin of the user, and a part of the insertion element 114, with the insertable portion 138 disposed therein, enters the body tissue. Thereafter, the insertion element 114 is retracted from the body tissue, in an upward retraction movement in FIG. 1, whereas the insertable portion 138 of the analytical sensor 136 remains in the body tissue. The housing 118 specifically may comprise, as shown in the embodiment of FIG. 1, a through hole 148 for receiving the insertion element 114 and in which, specifically, the through insertion element 114 or a part thereof, such as the cannula 142, may be guided and may move in an insertion direction, i.e., the downward direction in FIG. 1, and/or in a retraction direction, i.e., the upward direction in FIG. 1. The through hole 148 specifically may be at least partially formed or surrounded by the through hole part 128 of the housing 118. The circuit carrier 132, as an example, may surround the through hole 148, e.g., concentrically, as shown in FIG. 1. After insertion and after retraction of the insertion element 114 from the body tissue, wherein the insertion element 114 specifically may be entirely removed from the medical system 110, the housing 118 may rest, with an application side 150, directly or indirectly on the body surface of the user. For this purpose, the application side 150, as an example, may comprise at least one adhesive, such as at least one adhesive plaster, for attachment to the skin of the user.

The interior space 120 of the housing 118 specifically may be at least partially sealed from the surrounding of the medical device 112, specifically in order to avoid humidity or water to enter the interior space 120, thereby possibly deteriorating the functioning of the electronics unit 116 and/or the analytical sensor 136. The analytical sensor, however, is electrically connected to the electronics unit 116, with its electrical connection portion 140, as outlined above. In order to enable the insertable portion 138 of the analytical sensor 136 to protrude from the housing 118, the housing 118 comprises an opening 152, which may be surrounded by an opening rim 154 in the housing 118, such as an opening rim 154 partially formed by the lower housing part 122 and partially formed by the upper housing part 124, e.g., the through hole part 128 of the housing 118. As can be seen in FIG. 1, in the region of the opening 152, the analytical sensor 136 may be bent, e.g., by approximately 90°. Thus, the analytical sensor 136, within the housing 118, may essentially be oriented in a horizontal fashion, essentially parallel to the body surface of the user, whereas, outside the housing 114 and within the body tissue, the analytical sensor 136, specifically the insertable portion 138, may essentially be oriented in a non-horizontal fashion, e.g., in and essentially vertical fashion. In order to avoid a transfer of movement from the body tissue, via the insertable portion 136, onto the electrical connection portion 140 during use, the analytical sensor 136, within the housing 118, may fully or partially be fixated by adhesive, such as by fully or partially filling the housing 118 with adhesive, by gluing the analytical sensor 136 to the circuit carrier 132 or the like.

The bending as well as the gluing, however, may exert a considerable stress onto the analytical sensor 136, implying the risk of sensor deterioration or even failure. For this purpose, as will be shown in the context of the embodiments of FIGS. 2, 3, 4 and 5A and 5B, the medical device 112 comprises at least one guiding element 156 for guiding and protecting the analytical sensor 136, specifically in the region of the opening 152. The guiding element 156 is not shown in the overview of the medical system 110 in FIG. 1, since all of the embodiments of FIGS. 2, 3, 4 and 5A and 5B may be implemented in the embodiment of FIG. 1 as well as in other embodiments of medical systems of similar kind.

In a first embodiment, as shown in FIG. 2, the guiding element 156 comprises a lower guiding element 158 and an upper guiding element 160. These lower and upper guiding elements 158, 160 may be embodied as independent parts or may be embodied as inserts which form components of the housing 118. The housing 118 is mainly made of at least one housing material 162. Thus at least one housing material, as an example, may form the lower housing part 122 as well as the upper housing part 124. One or more housing materials 162 may be used, e.g., thermoplastic materials. The guiding element 156, contrarily, is made of at least one guiding material 170, which is softer than the housing material 162. As an example, while the housing material 162 may comprise or may consist of at least one hard thermoplastic material, the guiding material 170 may comprise or may consist of a soft material, specifically a flexible or deformable material, such as an elastomeric material and/or a foam or sponge material.

As an example, the housing material 162 may have a Shore D hardness of at least 40, specifically a Shore D hardness of 50 to 80. The guiding material 170 specifically may have a Shore A hardness of 0 to 70, specifically a Shore A hardness of 10 to 50. Exemplarily, the housing material 162 specifically may comprise at least one thermoplastic material. More specifically, the housing material 162 may be a thermoplastic material which may be selected from the group consisting of: polycarbonate (PC), exemplarily Makrolon 2458; acrylonitrile butadiene styrene copolymer (ABS). Exemplarily, the guiding material 170 specifically may comprise at least one elastomeric material. More specifically, the guiding material 170 may be selected from the group consisting of: a thermoplastic elastomer (TPE), exemplarily THERMOLAST® M TM6ADT; a silicone rubber (VMQ), exemplarily liquid silicone rubber (LSR).

The guiding element 156, in the embodiment shown in FIG. 2 may form inserts 164 in the housing 118. These inserts 164 may either be loosely inserted into the housing 118 or may be integrated by material connection, e.g., by multicomponent injection molding or insert molding, in the region of the opening 152. Thus, the inserts specifically may form opposing curved surfaces 166, 168, along which the curved part of the analytical sensor 136 is guided.

For assembly, the analytical sensor 136 may be inserted into the lower housing part 122 or baseplate, with the lower guiding element 158 underneath, loosely inserted or integrated into the lower housing part 122, the lower guiding element 158 forming a soft guiding part along the lower curved surface 166. Subsequently, the analytical sensor 136 may be covered, from above, with the at least one part 124, specifically the through hole part 128 with the upper guiding element 160 either loosely inserted therein or integrated therein, the upper guiding element 160 forming a soft guiding part along the upper curved surface 168. Thereby, the analytical sensor, in the opening 152 and, specifically, along the curved surfaces 166, 168, is surrounded by soft components, is sealed and guided along the curvature of the curved surfaces 166, 168.

In FIG. 3, a further embodiment of the guiding element 156 and its implementation into the medical device 112 is shown. The embodiment is basically similar to the embodiment in FIG. 2, with a lower guiding element 158 and an upper guiding element 160. In this embodiment, however, the lower guiding element 158 and the upper guiding element 160 are connected via a hinge 172 or a connecting part. Thus, as an example, the lower guiding element 158, as in the embodiment of FIG. 2, may be formed by an insert 164, either loosely inserted into the lower housing part 122 or integrated by material connection, with a guiding material 170 being softer than the housing material 162. The analytical sensor 136 is placed on top of the lower guiding element 158. Subsequently, the guiding element 156 is folded about hinge 172, indicated by folding direction 174 in FIG. 3, until the upper guiding element 160 rests on top of the analytical sensor 136. Again, as in the embodiment of FIG. 2, the analytical sensor, in the opening 152 and, specifically, along the curved surfaces 166, 168, is surrounded by soft components, is sealed and guided along the curvature of the curved surfaces 166, 168.

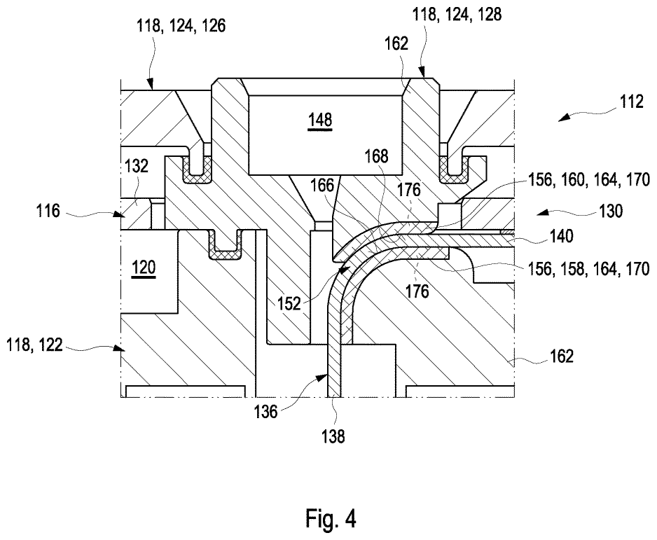

In FIG. 4, an embodiment is shown which is similar to the embodiment in FIG. 2. Again, lower and upper guiding elements 158, 160 are provided which are formed as inserts 164. As mentioned above in the context of the embodiments of FIG. 2, inserts 164 may be integrated into the lower and upper housing parts 122, 124, e.g., by multicomponent injection molding or insert molding. In the embodiment of FIG. 4, however, loose inserts are used which are loosely inserted into corresponding cavities 176 within the lower and upper housing parts 122, 124. Thus, again, as in FIG. 2, the analytical sensor 136 is sandwiched in between the lower housing part 122 with the lower guiding element 158 inserted therein, on the lower side, and the upper housing part 124 with the upper guiding element 160 inserted therein, so that the analytical sensor, at least in the region of the curved surfaces 166, 168, is specifically fully embedded in the soft guiding material 170.

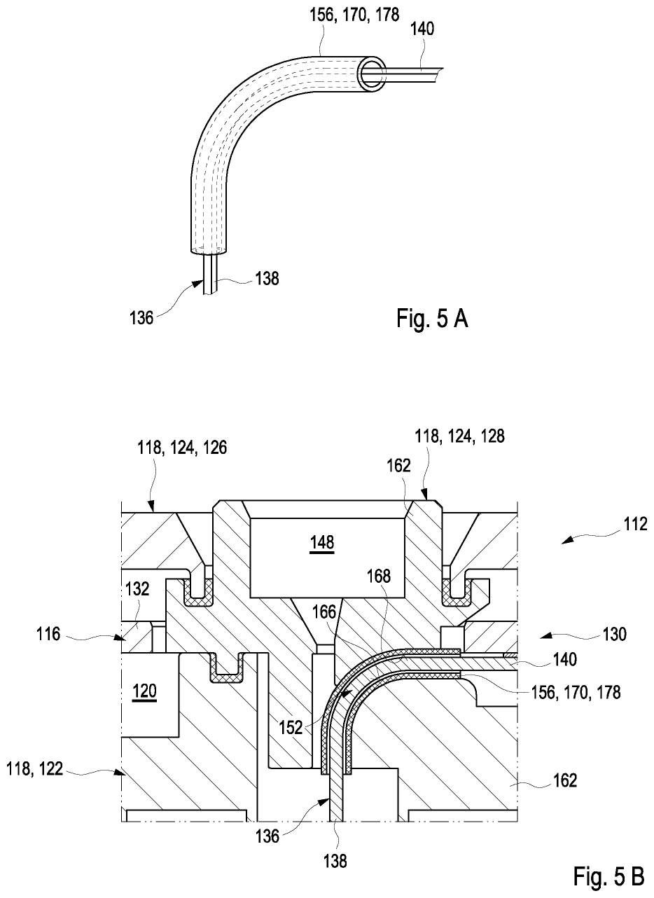

In FIGS. 5A and 5B, a slightly different concept of the guiding element 156 is shown, which may be used as an alternative or in addition to the embodiments shown in FIGS. 2, 3 and 4. In this embodiment, before insertion into the housing 118, the analytical sensor 136 is partially surrounded by the guiding element 156 forming a tube 178, as indicated in the detail view of FIG. 5A, showing the analytical sensor 136 and the tube 178 surrounding the same. The analytical sensor 136 may be sheathed by the tube 178 before inserting the analytical sensor into the housing 118. The tube 178 may fully or partially be made of the guiding material 170. FIG. 5B shows the implementation of the analytical sensor 136 with the tube 178 surrounding the same into the medical device 112. Thus, the tube 178 is placed around the analytical sensor 136 in the region of the curved surfaces 166, 168, such that the major part of the insertable portion 138 protruding into the body tissue, specifically the part carrying electrodes of the analytical sensor 136, as well as the electrical connection portion 140 remain free of the tube 178. As in all other embodiments, the size of the opening 152 may be chosen such that the analytical sensor 136, with the tube 178 surrounding the analytical sensor 136, fits into the opening 152, specifically in a press-fit manner, in order to further improve the sealing effect.

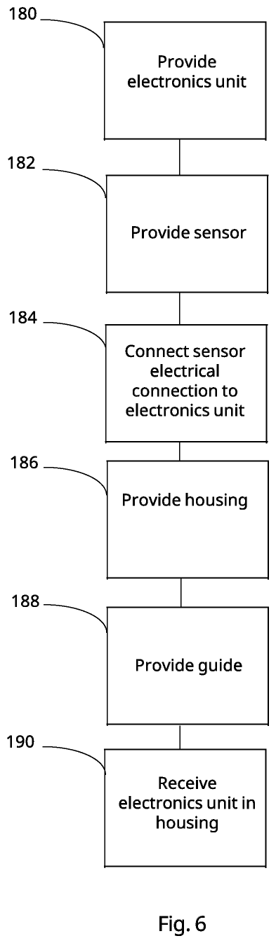

In FIG. 6, a flowchart of an embodiment of a method of assembling a medical device 112 is shown. The medical device 112 may, as an example, be embodied as shown in one of the embodiments of FIGS. 1 to 5B as discussed above. The method comprises the step shown in FIG. 6, which specifically may be performed in the given order.

However, a different order is also feasible. Further, it is possible to perform two or more or even all of the method steps in a fashion overlapping in time or at least partially simultaneously. It is further possible to perform each of the method steps only once or repeatedly. The method may comprise additional method steps which are not listed herein.

The method comprises the following steps:
a. providing at least one electronics unit 116 (step 180 in FIG. 6);
b. providing at least one analytical sensor 136 for transdermal insertion into a body tissue of a user, the analytical sensor 136 having an insertable portion 138 configured for at least partially being inserted into the body tissue and at least one electrical connection portion 140 (step 182 in FIG. 6);
c. electrically connecting the electrical connection portion 140 of the analytical sensor 136 to the electronics unit 116 (step 184 in FIG. 6);
d. providing at least one housing 118 being made of at least one housing material 162 (step 186 in FIG. 6);
e. providing at least one guiding element 156, the guiding element 156 at least partially being made of at least one guiding material 170 softer than the housing material 162 (step 188 in FIG. 6); and
f. receiving the electronics unit 116 in the housing 118, such that the analytical sensor 136, with at least the insertable portion 138, protrudes from at least one opening 152 in the housing 118, wherein the guiding element 156 at least partially surrounds the analytical sensor 136 at the opening 152 (step 182 in FIG. 6).

For details and options of the method steps, reference may be made to the description of the various embodiments of the medical device 112 and the various types of guiding elements 156 as discussed above. Other options, however, are also feasible.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 Medical system
112 Medical device
114 Insertion element
116 Electronics unit
118 Housing
120 Interior space
122 Lower housing part
124 Upper housing part
126 Upper shell
128 Through hole part
130 Control part
132 Circuit carrier
134 Electronic component
136 Analytical sensor
138 Insertable portion
140 Electrical connection portion
142 Cannula
144 Receptacle
146 Sharp
148 Through hole
150 Application side
152 Opening
154 Opening rim
156 Guiding element
158 Lower guiding element
160 Upper guiding element
162 Housing material
164 Insert
166 Lower curved surface
168 Upper curved surface
170 Guiding material
172 Hinge
174 Folding direction
176 Cavity
178 Tube
180 Providing electronics unit
182 Providing analytical sensor
184 Electrically connecting electrical connection portion to electronics unit
186 Providing housing
188 Providing guiding element
190 Receiving electronics unit in housing

What is claimed is:

1. A medical device for detecting at least one physiological parameter of a user, the medical device comprising:
an electronics unit;
an analytical sensor configured for transdermal insertion into body tissue of a user, the analytical sensor having an insertable portion configured for at least partially being inserted into the body tissue and an electrical connection portion electrically connected to the electronics unit;
a housing made of a housing material, the housing receiving the electronics unit, wherein at least the insertable portion protrudes from an opening in the housing; and
a guide at least partially made of a guiding material softer than the housing material, the guide at least partially surrounding the analytical sensor at the opening.

2. The medical device according to claim 1, wherein the guide is (i) separate from the housing or (ii) fully or partially integrated into the housing, with the housing being a multicomponent housing comprising at least the housing material and the guiding material.

3. The medical device according to claim 1, wherein the guide comprises an insert that is inserted into the housing.

4. The medical device according to claim 3, wherein the insert comprises at least one of:
a tube partially surrounding the analytical sensor;
a sandwich comprising at least one upper insert and at least one lower insert, with the analytical sensor embedded between the upper insert and the lower insert;
a foldable insert having at least one insert base component and at least one foldable insert component, with the foldable insert component being foldable over the analytical sensor such that in a folded state the analytical sensor is embedded between the insert base component and the foldable insert component.

5. The medical device according to claim 1, wherein the guide comprises a curved surface over which the analytical sensor is guided.

6. The medical device according to claim 5, wherein the guide comprises at least two opposing curved surfaces, wherein the analytical sensor is guided between the two opposing curved surfaces.

7. The medical device according to claim 1, wherein the housing material has a Shore D hardness of at least 40.

8. The medical device according to claim 1, wherein the guiding material has a Shore A hardness of 70 or less.

9. The medical device according to claim 1, wherein the housing material has a Shore D hardness of about 80 and the guiding material has a Shore A hardness of 20 to 50.

10. The medical device according to claim 1, wherein the housing material comprises at least one thermoplastic material.

11. The medical device according to claim 1, wherein the housing material is a thermoplastic material which is selected from the group consisting of polycarbonate (PC) and acrylonitrile butadiene styrene copolymer (ABS).

12. The medical device according to claim 1, wherein the guiding material comprises at least one elastomeric material.

13. The medical device according to claim 1, wherein the guiding material is selected from the group consisting of thermoplastic elastomer (TPE) and silicone rubber (VMQ).

14. The medical device according to claim 1, wherein the analytical sensor is bent, wherein the guide at least partially surrounds a bent portion of the analytical sensor.

15. A medical system, comprising:
the medical device according to claim 1;
an insertion element, wherein the insertable portion protruding from the opening in the housing is at least partially received in the insertion element.

16. A method of assembling a medical device for detecting at least one physiological parameter of a user, the method comprising:
a. providing an electronics unit;
b. providing an analytical sensor configured for transdermal insertion into body tissue, the analytical sensor having an insertable portion configured for at least partially being inserted into the body tissue and an electrical connection portion;
c. electrically connecting the electrical connection portion of the analytical sensor to the electronics unit;
d. providing a housing made of a housing material;
e. providing a guide at least partially made of a guiding material softer than the housing material; and
f. receiving the electronics unit in the housing such that the at least the insertable portion of the analytical sensor protrudes from an opening in the housing, wherein the guide at least partially surrounds the analytical sensor at the opening.
